(19) United States
(12) Patent Application Publication
(10) Pub. No.: US 2016/0331962 A1
(43) Pub. Date: Nov. 17, 2016

(54) METHODS FOR IMPROVING A PATIENT'S IMMUNE RESPONSE

(71) Applicant: Ohio State Innovation Foundation, Columbus, OH (US)

(72) Inventors: Jan Schwab, Columbus, OH (US); Phillip Popovich, Columbus, OH (US); Ali R. Rezai, Columbus, OH (US)

(21) Appl. No.: 15/155,466

(22) Filed: May 16, 2016

Related U.S. Application Data
(60) Provisional application No. 62/162,261, filed on May 15, 2015.
(60) Provisional application No. 62/180,760, filed on Jun. 17, 2015.

(51) Int. Cl. A61N 1/36; A61M 5/142; A61M 5/172; A61N 1/05

(52) U.S. Cl. CPC A61N 1/3606; A61N 1/0551; A61N 1/36139; A61M 5/14276; A61M 5/1723; A61M 2210/1003; A61M 2210/0693

(57) ABSTRACT

Methods for modulating immune function in a patient are provided. Methods include improving an immune response in a patient suffering from a condition resulting or caused by a deficient immune system. A step in such a method includes positioning a therapy delivery device on a neural target site of a maladaptive sympathetic reflex of the patient. An additional step includes activating the therapy delivery device to deliver a therapy signal to the neural target site to improve the patient's immune response.

### RELATED APPLICATIONS

This application claims priority to U.S. Provisional Patent Application Ser. No. 62/162,261, filed May 15, 2015, and U.S. Provisional Application No. 62/180,760, filed on Jun. 17, 2015. The entirety of both applications is hereby incorporated by reference for all purposes.

### TECHNICAL FIELD

The present disclosure generally relates to improving a patient's immune response by neuromodulation and other forms of therapy.

### BACKGROUND

The central nervous system (CNS) controls the immune system by several pathways, including being hardwired to the autonomic nervous system (ANS). Sensors within the central and peripheral autonomic system (PNS) relay information about the status of the immune system. Disruption of coordinated CNS-immune system interaction after injury or disease can result in an abrupt and drastic decrease in immune function.

A need exists for methods to balance or enhance sympathetic tone in patients suffering from immune-related diseases or conditions.

### SUMMARY

The present disclosure relates generally to methods for improving a patient's immune response. In an embodiment, a method is provided for improving an immune response in a patient suffering from a condition resulting or caused by a deficient immune system. The method comprises positioning a therapy delivery device on a neural target site of a maladaptive sympathetic reflex of the patient and activating the therapy delivery device to deliver a therapy signal to the neural target site. Such therapy signal improves the patient's immune response. Another method comprises positioning a therapy delivery device on an autonomic neural target site that innervates an endocrinological tissue, a lymphatic tissue, an immune system organ involved in an immune response in the patient. The method further comprises blocking neural conduction in the autonomic neural target site to improve the patient's immune response.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a process flow diagram illustrating a method for modulating immune function in a patient according to an embodiment of the present disclosure;

FIG. 2 is a schematic illustration of the levels of integration of the sympathetic (autonomic) nervous system and their implications after CNS injury according to an embodiment of the present disclosure: (A) Schematized supraspinal centers (brain-stem and brain) versus spinal cord mapping to neuroendocrine and immunological effector organs; and (B) Traumatic brain injury (TBI), stroke and subarachnoid hemorrhage are prevalent causes for damage to supraspinal centers. Spinal cord injury damages spinal preganglionic neurons. As a consequence of injury at all depicted anatomical locations—either of traumatic of non-traumatic origin—disinhibited sympathetic nerve activity (SNA) will enter neuroendocrine and immunological effector organs and result in immune suppression;

FIG. 3 is a process flow diagram illustrating a method for modulating immune function in a mammal according to another embodiment of the present disclosure; and

FIG. 4 is a schematic illustration of the neurogenic pathophysiology and targets of maladaptive sympathetic signaling perpetuating functional spinal cord injury induced immune depression syndrome after spinal cord injury.

### DETAILED DESCRIPTION

The present disclosure is generally directed to improving a patient's immune function. Improving a patient's immune function includes normalizing, restoring, regulating, increasing activity and function, or decreasing activity of function so that the patient's immune system is modulated to improve hyperactive or hypoactive immune systems.

As used herein with respect to a described element, the terms “a,” “an,” and “the” include at least one or more of the described element unless otherwise indicated. Further, the term “or” and “and” refer to “and/or” unless otherwise indicated. In addition, it will be understood that when an element is referred to as being “on,” “attached” to, “connected” to, “coupled” with, “contacting,” “in fluid communication with,” etc., another element, it can be directly on, attached to, connected to, coupled with, contacting, or in fluid communication with the other element or intervening elements may also be present. In contrast, when an element is referred to as being, for example, “directly on,” “directly attached” to, “directly connected” to, “directly coupled” with, “directly contacting,” or in “direct fluid communication with,” another element, there are no intervening elements present. It will also be appreciated by those of skill in the art that references to an element that is disposed “adjacent” another element may have portions that overlap or underlie the adjacent element.

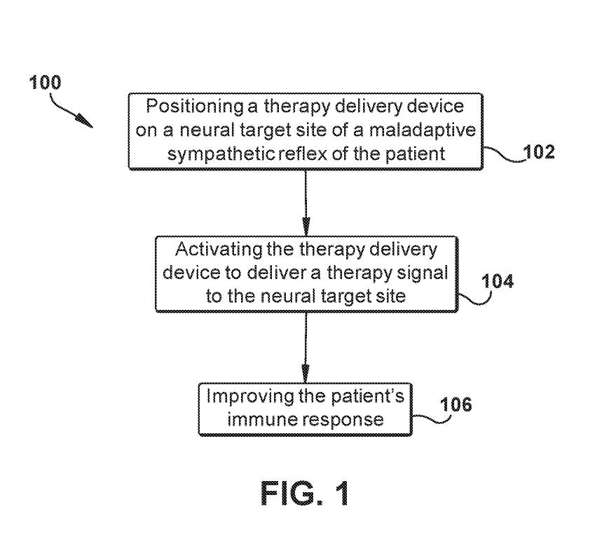

Referring to FIG. 1, in general, a method 100 of improving an immune response in a patient suffering from a condition resulting or caused by a deficient immune system comprises positioning a therapy delivery device on a neural target site of a maladaptive sympathetic reflex of the patient 102. The therapy delivery device is activated to deliver a therapy signal to the neural target site 104 to improve the patient's immune response 106. Preferably, the patient is a human being.

The neural target site can be a PNS or a CNS structure anatomically relevant to maladaptive sympathetic reflexes. Preferably, the neural target site innervates an endocrinological or lymphatic tissue or organ involved in the immune response of the patient. In certain embodiments, the neural target site is a target site of the ANS. For example, the target site can be the celiac ganglion, the superior mesenteric ganglion, the aorticorenal ganglion, the renal plexus, the inferior mesenteric ganglion, the superior hypogastric ganglion, the lumbar plexus, the celiac plexus, the splenic nerve, sympathetic trunk, splanchnic nerves and their input and output nervous system structures. In other embodiments, the target site can be the spinal cord (including the thoracic, cervical, and lumbar segments), a dorsal root ganglion, preganglionic fibers, or post ganglionic fibers, an adrenal nerve. The target site can be other central regions such as within the forebrain, including the hypothalamus; the insular cortex; the nucleus coeruleus; the prefrontal cortex and brainstem including the midbrain-pons-medulla circuitry that control output from the spinal cord to immune organs. Such central structures or neural regions directly “map” to spinal cord sympathetic preganglionic neurons and subsequently to peripheral target sites such as ganglia controlling immune organs.

In any of the methods described herein and as described in more detail below, pharmacotherapy can be used in combination as adjuvant therapy together with neuromodulation. Pharmacotherapy can include medications that shift the patient's autonomic tone such as, for example, beta blockers, angiotensin converting enzyme (ACE) blockers, anti-spasticity, and anti-convulsive drugs. Pharmacotherapies can also include medications or combinations of medications that can mimic or replace the effects of neuromodulation on the immune system. Such drugs can be used alone or in combination with neuromodulation. Such drugs include, for example, select beta blockers that target B1 and B2 adrendergic receptors found on immune cells or glucocorticoid receptor antagonists.

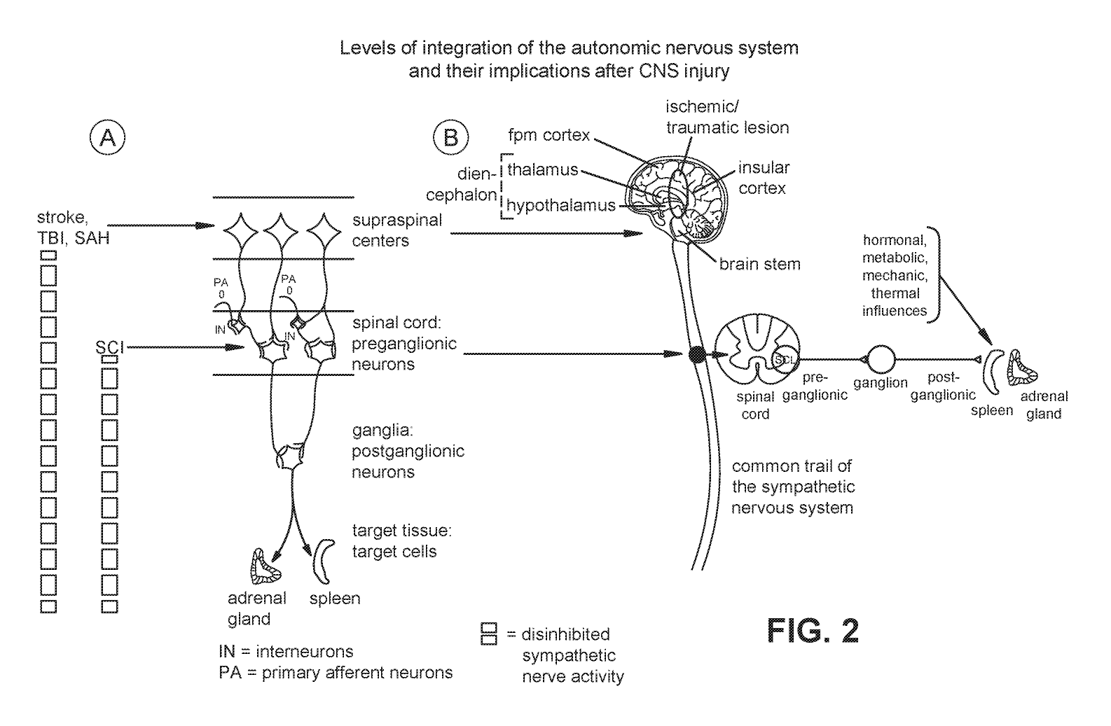

A maladaptive sympathetic reflex is disinhibited sympathetic nerve activity that enters the lymphatic organs, endocrine organs, such as the spleen and/or the adrenal gland, immune system organ or immune system cells as illustrated in FIG. 2. A maladaptive reflex includes a sympathetic anti-inflammatory reflex, Sympathico-Babinski, disinhibited spinally generated sympathetic activity, and paroxysmal sympathetic activity. These conditions generally act on the autonomic nervous system (ANS) causing a systemic immune-suppression or immune-activation. A maladaptive sympathetic reflex can be present in a patient with an imbalanced immune system including a hyperactive or hypoactive immune system. A maladaptive sympathetic reflex can be present after CNS injury includes injury to the brain, such as stroke, traumatic brain injury (TBI), subarachnoid heamorrhage (SAH), and spinal cord injury (SCI), and other CNS or spinal cord or nervous system damage/injury, such as damage to nerves. Injury can be due to acute or chronic degenerative conditions. Chronic degeneration conditions include injury to the brain or spinal cord by compressive tumor growth but also degenerative myelopathy and neurodegenerative disease.

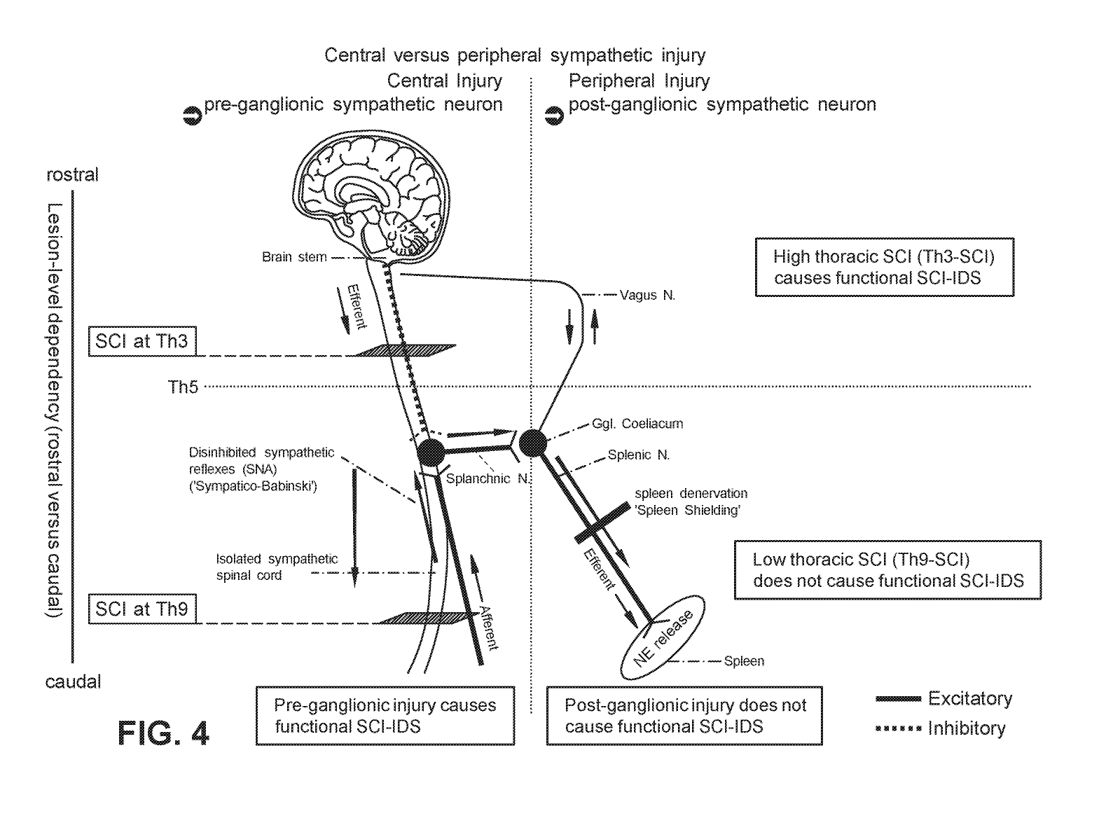

Without wishing to be bound by theory, FIG. 4 schematically illustrates the neurogenic pathophysiology and targets of maladaptive sympathetic signaling perpetuating functional SCI-IDS after SCI. Under physiological conditions, excitatory sympathetic signals are controlled by supraspinal inhibition. After SCI, loss of supraspinal control leads to spinally generated sympathetic nerve activity (SNA). SNA originates below SCI injury and leads to maladaptive efferent sympathetic activity signaling to the spleen, via the splenic or splanchnic nerve and the adrenal gland. It is able to culminate in large excitatory spinal sympathetic reflexes. Spinal sympathetic reflexes occur in analogy to pathological Babinski motor reflexes, caused by a loss of supraspinal (bulbospinal) tonic inhibition (“Sympathetic-Babinski). Again, without wishing to be bound by theory, disinhibited spinal generated sympathetic nerve activity after high thoracic (Th3) SCI can lead to ad hoc induction of excitatory SNA burst entering the splenic nerve and adrenal gland associated with increased levels of norepinephrine (NE) in the circulation and the spleen, which in turn causes apoptosis of immune cells in the spleen and other lymphoid organs. This results in a decrease of spleen size and an elevated susceptibility to infection. Blocking SNA signaling (“shielding”) by preceding peripheral denervation of the splenic nerve ameliorates functional SCI-IDS and bacterial load after Th3-SCI. Moreover, the peripheral splenic nerve can be a target for immunomodulation after SCI in order to restore impaired host defense after SCI (SCI-IDS) by blocking SNA to the spleen. As such, spleen and adrenal gland shielding can be an interventional strategy to ameliorate functional SCI-IDS to prevent infections in patients at risk.

Again, without wishing to be bound by theory, it is believed that enhancing the maladaptive sympathetic reflex may also artificially immune-suppress a patient and may attenuate autoimmune diseases affecting all systems of the body. Those systems comprise but are not limited to the nervous system (peripheral and central), bone, cartilage, bronchial system/lung, pancreas, liver, and hematological systems. Blocking sympathetic activation reactive to stress responses may harness a patient's immune defense system against cancer. Other conditions causing immune suppression and elevated risk for infections are listed in Table I below.

TABLE I
Term

Epileptogenic
tonic decerebrate seizures
autonomic seizures with decerebrate seizures
diencephalic (autonomic) seizures/epilepsy/fits
autonomic sympathetic seizures
Structural
brain stem attack
hypothalamic-midbrain dysregulation syndrome
acute midbrain syndrome
acute hypothalamic instability
hypothalamic storm
Clinically Descriptive
paroxysmal hypertension associated with diaphoresis
(central) autonomic dysfunction/syndrome
hyperthermic syndrome
neuroleptic malignant-like syndrome
autonomic storms/storming
autonomic hyperactivity
dysautonomia
(central) autonomic instability
hyperpyrexia with sustained muscle contraction
autonomic dysregulation
paroxysmal sympathetic storms
sympathetic storms/storming
autonomic disorder (and spasticity)
neuro storm
paroxysmal autonomic instability with dystonia
hyperadrenergic state
paroxysmal autonomic instability
dysautonomic crises
arc de cercie and dysautonomia
storming: transient autonomic dysfunction
paroxysmal sympathetic hyperactivity
hypermetabolic paroxysmal dysautonomia

Accordingly, in certain embodiments, the therapy signal blocks neural conduction in the neural target site. Such blocking of neural conduction can reduce or balance the patient's sympathetic tone to improve the patient's immune response. In certain embodiments, the patient is suffering from sepsis, a central nervous system injury, antibiotic resistance, compensatory anti-inflammatory response syndrome (CARS), chronic inflammatory response syndrome (ORS), spinal cord injury-induced immune depression syndrome (SCI-IDS), traumatic brain injury-induced immune depression syndrome (TBI-IDS), combined immune deficiency syndrome (CIDS), or other immune deficiencies.

In certain embodiments, the therapy signal stimulates neural conduction in the neural target site. Such stimulation can enhance the patient's sympathetic tone to immune-suppress the patient, suppress the patient's baseline sympathetic tone to suppress disease-associated increases in immune function, or increase activity of the patient's intact sympathetic circuitry to suppress the patient's immune function. Such stimulation can be used in circumstances where the patient has undergone organ transplantation (including bone marrow transplantation); suffers from an autoimmune disorder, including multiple sclerosis, rheumatoid arthritis, myasthenia gravis or myositis; suffers from corticosteroid sparing including osteoporosis or cortisone-induced psychosis; or suffers from other adverse effects from immune suppressive therapies.

Other conditions include hyper-immune disorders including multiple sclerosis and rheumatoid arthritis, and allergic conditions. Conditions also include hypo-active immune system disorders such as cancers or infections.

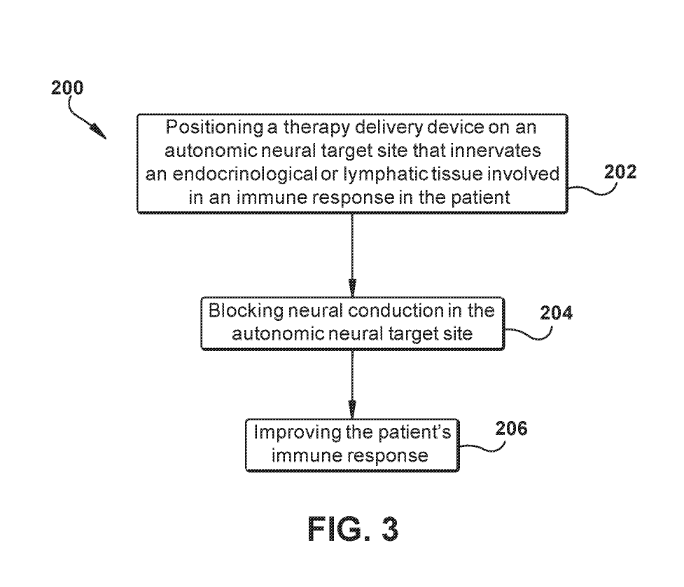

Referring to FIG. 3, in other embodiments, the present disclosure provides a method of improving an immune response in a patient suffering from a condition resulting or caused by a deficient immune system 200. The method includes positioning a therapy delivery device on an autonomic neural target site that innervates an endocrinological or lymphatic tissue involved in an immune response in the patient 202. The method further includes blocking neural conduction in the autonomic neural target site 204 and improving the patient's immune response 206. The autonomic neural target site can be the celiac ganglion, the superior mesenteric ganglion, the aorticorenal ganglion, the renal plexus, the inferior mesenteric ganglion, the superior hypogastric ganglion and the lumbar plexus. The condition can be, for example, sepsis, a central nervous system injury, antibiotic resistance, compensatory anti-inflammatory response syndrome (CARS), chronic inflammatory response syndrome (CIRS), a spinal cord injury-induced immune depression syndrome (SCI-IDS), traumatic brain injury-induced immune depression syndrome (TBI-IDS), or combined immune deficiency syndrome (CIDS).

In certain aspects, the present disclosure includes various therapy delivery devices (not shown) and related systems configured to modulate a patient's immune response. In some instances, therapy delivery devices may be positioned directly on a target nerve, neuron or nerve structure. In other instances, therapy delivery devices may be positioned below the skin of a mammal but not directly on a target nerve, neuron or nerve structure. In further instances, therapy delivery devices may comprise an external device, e.g., positioned in a lumen adjacent a target nerve, neuron or nerve structure. In still further instances, therapy delivery devices can include an external device, e.g., positioned on the skin of a mammal adjacent a target nerve, neuron or nerve structure. Therapy delivery devices can be temporarily or permanently implanted within, on, or otherwise associated with a mammal in need of immune function modulation.

Therapy delivery devices can be configured or programmed to deliver various types of therapy signals to a target nerve, neuron or nerve structure. For example, therapy delivery devices can be configured or programmed to deliver only electrical energy, only a pharmacological or biological agent, or a combination thereof. In one example, therapy delivery devices can comprise at least one electrode and an integral or remote electrical energy generator (not shown), which is in electrical communication with the one or more electrodes and configured to produce one or more electrical signals (or pulses). In another example, therapy delivery devices can include a pharmacological or biological agent reservoir, a pump, and a fluid dispensing mechanism. Non-limiting examples of pharmacological and biological agents can include chemical compounds, drugs, nucleic acids, polypeptides, stem cells. The therapy delivery device can also be configured or programmed to deliver various energy and/or biological forms, such as ultrasound, radiofrequency (continuous or pulsed), magnetic waves, cryotherapy, heat, or optogenetic therapy.

In yet another example, therapy delivery devices can be configured or programmed to deliver magnetic nerve stimulation with desired field focality and depth of penetration. One skilled in the art will appreciate that combinations of the therapy delivery devices above configurations are also included within the scope of the present disclosure.

In some instances, therapy delivery devices can include a stimulator (or inhibitor), such as an electrode or electrical lead, a controller or programmer, and one or more connectors for connecting the stimulating (or inhibiting) device to the controller. In further describing representative electrodes, which are described in the singular, it will be apparent that more than one electrode may be used as part of a therapy delivery device. Accordingly, the description of a representative electrode suitable for use in the therapy delivery devices of the present disclosure is applicable to other electrodes that may be employed.

An electrode can be controllable to provide output signals that may be varied in voltage, frequency, pulse-width, current and intensity. The electrode can also provide both positive and negative current flow from the electrode and/or is capable of stopping current flow from the electrode and/or changing the direction of current flow from the electrode. In some instances, therapy delivery devices can include an electrode that is controllable, i.e., in regards to producing positive and negative current flow from the electrode, stopping current flow from the electrode, changing direction of current flow from the electrode, and the like. In other instances, the electrode has the capacity for variable output, linear output and short pulse-width. In other instances, the electrode can comprise a coil configured to deliver magnetic stimulation.

The electrical energy generator can comprise a battery or generator, such as a pulse generator that is operatively connected to the electrode. For example, the electrical energy generator can include a battery that is rechargeable by inductive coupling. The electrical energy generator may be positioned in any suitable location, such as adjacent the electrode (e.g., implanted adjacent the electrode), or a remote site in or on the mammal's body or away from the mammal's body in a remote location. An electrode may be connected to the remotely positioned electrical energy generator using wires, e.g., which may be implanted at a site remote from the electrode or positioned outside the mammal's body. In one example, implantable electrical energy generators analogous to a cardiac pacemaker may be used.

The electrical energy generator can control the pulse waveform, the signal pulse width, the signal pulse frequency, the signal pulse phase, the signal pulse polarity, the signal pulse amplitude, the signal pulse intensity, the signal pulse duration, and combinations thereof of an electrical signal. The electrical energy generator may be programmed to convey a variety of currents and voltages to one or more electrodes and thereby modulate the activity of a nerve, neuron, or nerve structure. The electrical energy generator may be programmed to control numerous electrodes independently or in various combinations as needed to provide stimulation. In some instances, an electrode may be employed that includes its own power source, e.g., which is capable of obtaining sufficient power for operation from surrounding tissues in the mammal's body, or which may be powered by bringing a power source external to the mammal's body into contact with the mammal's skin, or which may include an integral power source.

In other instances, an electrical signal may be constant, varying and/or modulated with respect to the current, voltage, pulse-width, cycle, frequency, amplitude, and so forth. For example, a current may range from about 0.001 to about 1000 microampere (mA) and, more specifically, from about 0.1 to about 100 mA. Similarly, the voltage may range from about 0.1 millivolt to about 25 volts, or about 0.5 to about 4000 Hz, with a pulse-width of about 10 to about 1000 microseconds. The type of stimulation may vary and involve different waveforms known to the skilled artisan. For example, the stimulation may be based on the H waveform found in nerve signals (i.e., Hoffinan Reflex). In another example, different forms of interferential stimulation may be used.

To increase activity in a portion of the neural target site such as the ANS, for example, voltage or intensity may range from about 1 millivolt to about 1 volt or more, e.g., 0.1 volt to about 50 volts (e.g., from about 0.2 volt to about 20 volts), and the frequency may range from about 1 Hz to about 2500 Hz, e.g., about 1 Hz to about 1000 Hz (e.g., from about 2 Hz to about 100 Hz). In some instances, pure DC voltages may be employed. The pulse-width may range from about 1 microsecond to about 2000 microseconds or more, e.g., from about 10 microseconds to about 2000 microseconds (e.g., from about 15 microseconds to about 1000 microseconds). The electrical signal may be applied for at least about 1 millisecond or more, e.g., about 1 second (e.g., about several seconds). In some instances, stimulation may be applied for as long as about 1 minute or more, e.g., about several minutes or more (e.g., about 30 minutes or more).

To decrease activity in a portion of the neural target site including the ANS, for example, voltage or intensity may range from about 1 millivolt to about 1 volt or more, e.g., 0.1 volt to about 50 volts (e.g., from about 0.2 volt to about 20 volts), and the frequency may range from about 1 Hz to about 2500 Hz, e.g., about 50 Hz to about 2500 Hz. In some instances, pure DC voltages may be employed. The pulse-width may range from about 1 microseconds to about 2000 microseconds or more, e.g., from about 10 microseconds to about 2000 microseconds (e.g., from about 15 microseconds to about 1000 microseconds). The electrical signal may be applied for at least about 1 millisecond or more, e.g., about 1 second (e.g., about several seconds). In some instances, the electrical energy may be applied for as long as about 1 minute or more, e.g., about several minutes or more (e.g., about 30 minutes or more may be used).

The electrode may be mono-polar, bipolar or multi-polar. Further, the electrode (and any wires and optional housing materials) can be made of inert materials, such as silicon, metal, plastic and the like. In one example, a therapy delivery device can include a multi-polar electrode having about four exposed contacts (e.g., cylindrical contacts).

A controller or programmer may also be associated with a therapy delivery device. A programmer, for example, can include one or more microprocessors under the control of a suitable software program. Other components of a programmer, such as an analog-to-digital converter, etc., will be apparent to those of skill in the art.

Therapy delivery devices can be pre-programmed with desired stimulation parameters. Stimulation parameters can be controllable so that an electrical signal may be remotely modulated to desired settings without removal of the electrode from its target position. Remote control may be performed, e.g., using conventional telemetry with an implanted electric signal generator and battery, an implanted radiofrequency receiver coupled to an external transmitter, and the like. In some instances, some or all parameters of the electrode may be controllable by the subject, e.g., without supervision by a physician. In other instances, some or all parameters of the electrode may be automatically controllable by a programmer or controller comprising the therapy delivery device.

The therapy delivery device can be configured for different forms of placement, insertion or implantation. This includes, for example, direct stimulation, epidural stimulation, and transvascular stimulation.

In one example, the therapy delivery device can be configured for percutaneous placement or implantation. In this instance, the therapy delivery device can comprise one or more implantable electrodes shaped or configured, for example, as a wire, a rod, a filament, a ribbon, a cord, a tube, a formed wire, a flat strip, or a combination thereof. In one example, one or more of the electrodes can comprise a laminotomy electrode array. Laminotomy electrodes, for example, generally have a flat paddle configuration and typically possess a plurality of electrodes (e.g., 2, 3, 4 or more) arranged on the paddle. The arrangement of electrodes on the paddle may be in rows and columns, staggered, spaced, circular, or any other arrangement that will position the electrodes for optimal delivery of electrical energy. The one or more implantable electrodes may be controlled individually, in series, in parallel, or any other manner desired. Once implanted, the implantable electrode(s) may be held in position using any method known to the skilled artisan, such as stitches, epoxy, tape, glue, sutures, or a combination thereof.

In another example, the device can be configured for transvascular placement or implantation. For example, the therapy delivery device can be placed across the azygous vein as or another venous and arterial system adjacent to the neural structure innervating or influencing the patient's immune function.

In another example, the therapy delivery device can be configured for intravascular or intraluminal placement or implantation. In some instances, a therapy delivery device configured for intravascular or intraluminal placement or implantation can be configured in an identical or similar manner as the expandable electrode disclosed in U.S. patent application Ser. No. 11/641,331 to Greenberg et al. (hereinafter, “the '331 application”).

In yet another example, the therapy delivery device can be configured for transcutaneous neuromodulation. In some instances, transcutaneous neuromodulation can include positioning an electrode on a skin surface so that a therapy signal can be delivered to a target nerve, neuron, or nerve structure. In other instances, transcutaneous neuromodulation can include positioning an electrode, without penetrating the skin of the subject and without necessarily contacting the electrode with the skin surface, so that a therapy signal can be delivered to a target nerve, neuron, or nerve structure. Transcutaneous neuromodulation can additionally include partially transcutaneous methods (e.g., using a fine, needle-like electrode to pierce the epidermis). In another example, a surface electrode can be placed into electrical contact with a nerve, neuron, or nerve structure (e.g., of the ANS) associated with immune function. Generally, an electrical signal used for transcutaneous neuromodulation may be constant, varying and/or modulated with respect to the current, voltage, pulse-width, cycle, frequency, amplitude, and so forth (e.g., the current may be between about 1 to 100 microampere), about 10 V (average), about 1 to about 1000 Hz, with a pulse-width of about 250 to about 500 microseconds.

Devices for transcutaneous neuromodulation can have a variety of configurations. In some instances, a transcutaneous therapy delivery device may be configured as a belt or strap having at least one electrode operably attached thereto. Alternatively, a transcutaneous therapy delivery device may be configured as an adhesive patch having at least one electrode operably attached thereto.

Therapy delivery devices can be part of an open- or closed-loop system. In an open-loop system, for example, a physician or subject may, at any time, manually or by the use of pumps, motorized elements, etc., adjust treatment parameters, such as pulse amplitude, pulse-width, pulse frequency, duty cycle, dosage amount, type of pharmacological or biological agent, etc. Alternatively, in a closed-loop system, treatment parameters (e.g., electrical signals) may be automatically adjusted in response to a sensed physiological parameter or a related symptom indicative of the extent of immune function. In a closed-loop feedback system, a sensor (not shown) that senses a physiological parameter associated with immune function can be utilized. More detailed descriptions of sensors that may be employed in a closed-loop system, as well as other examples of sensors and feedback control techniques that may be employed as part of the present disclosure are disclosed in U.S. Pat. No. 5,716,377.

It should be appreciated that incorporating the therapy delivery device as part of a closed-loop system can include placing or implanting a therapy delivery device on or within a mammal at a nerve target, sensing a physiological parameter associated with immune function, and then activating the therapy delivery device to apply a therapy signal to adjust application of the therapy signal to the nerve target in response to the sensor signal to modulate immune function. In certain instances, the physiological parameter is associated with a deficient immune system such as a hypoactive or hyperactive immune system. In some instances, such physiological parameters can include any characteristic or function associated with immune function, such skin temperature, protein concentrations, heart rate, blood pressure, biomarkers of the immune system or autonomic nervous system, electrochemical gradients, electrolytes, laboratory values, body temperature, and vital signs.

From the above description of the present disclosure, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

The following is claimed:

1. A method of improving an immune response in a patient suffering from a condition resulting or caused by a deficient immune system comprising:
positioning a therapy delivery device on a neural target site of a maladaptive sympathetic reflex of the patient;
activating the therapy delivery device to deliver a therapy signal to the neural target site; and
improving the patient's immune response.

2. The method of claim 1, wherein the neural target site innervates an endocrinological or lymphatic tissue involved in the immune response of the patient.

3. The method of claim 1, wherein the therapy signal blocks neural conduction in the neural target site.

4. The method of claim 3, wherein improving the patient's immune response comprises reducing or balancing the patient's sympathetic tone to improve the patient's immune response.

5. The method of claim 4, wherein the condition is selected from the group consisting of sepsis, a central nervous system injury, antibiotic resistance, compensatory anti-inflammatory response syndrome (CARS), chronic inflammatory response syndrome (CIRS), spinal cord injury-induced immune depression syndrome (SCI-IDS), traumatic brain injury-induced immune depression syndrome (TBI-IDS), combined CNS-injury induced immune deficiency syndrome (CIDS), and other immune deficiencies.

6. The method of claim 1, wherein the therapy signal stimulates neural conduction in the neural target site.

7. The method of claim 6, wherein improving the patient's immune response comprises enhancing the patient's sympathetic tone to immune-suppress the patient.

8. The method of claim 6, wherein improving the patient's immune response comprises suppressing the patient's baseline sympathetic tone to suppress disease-associated increases in immune function.

9. The method of claim 6, wherein improving the patient's immune response comprises increasing activity of the patient's intact sympathetic circuitry to suppress the patient's immune function.

10. The method of claim 6, wherein the condition is organ transplantation, an autoimmune disease, corticosteroid sparing, or other adverse effects from immune-suppressive therapies.

11. The method of claim 10, wherein the autoimmune disease is multiple sclerosis, rheumatoid arthritis, myasthenia gravis, or myositis.

12. The method of claim 6, wherein the condition is a hyper-allergic state.

13. The method of 6, wherein the condition is osteoporosis or cortisone-induced psychosis.

14. The method of claim 1, wherein the neural target site is a target site of the peripheral nervous system (PNS).

15. The method of claim 14, wherein the target site of the PNS is a target site of the autonomic nervous system (ANS).

16. The method of claim 15, wherein the target site of the ANS is selected from the group consisting of the celiac ganglion, the superior mesenteric ganglion, the aorticorenal ganglion, the renal plexus, the inferior mesenteric ganglion, the superior hypogastric ganglion, the lumbar plexus, the splenic nerve, the splanchnic nerve, and the sympathetic trunk.

17. The method of claim 1, wherein the neural target site is a target site of the central nervous system (CNS).

18. The method of claim 1, wherein the mammal is a human.

19. The method of claim 1, further comprising the steps of:
sensing a physiological parameter associated with the deficient immune system;
generating a sensor signal based on the physiological parameter; and
activating the therapy delivery device to adjust application of the therapy signal to the neural target site in response to the sensor signal to improve the patient's immune response.

20. The method of claim 19, wherein the deficient immune system is a hyperactive or hypoactive immune system.

21. The method of claim 19, wherein the physiological parameter is a biomarker of the patient's immune system or autonomic nervous system.

22. The method of claim 1, wherein the therapy delivery device is an electrical lead.

23. The method of claim 1, wherein the therapy delivery device is a catheter or drug pump.

24. The method of claim 1, further comprising administering a pharmacological agent that shifts the patient's autonomic tone.

25. A method of improving an immune response in a patient suffering from a condition resulting or caused by a deficient immune system comprising:
positioning a therapy delivery device on an autonomic neural target site that innervates an endocrinological, lymphatic tissue, or an immune system organ involved in an immune response in the patient;
blocking neural conduction in the autonomic neural target site; and
improving the patient's immune response.

26. The method of claim 25, wherein the autonomic neural target site is selected from the group consisting of the celiac ganglion, the superior mesenteric ganglion, the aorticorenal ganglion, the renal plexus, the inferior mesenteric ganglion, the superior hypogastric ganglion, the lumbar plexus, the splenic nerve, the splanchnic nerve, and the sympathetic trunk.

27. The method of claim 25, wherein the condition is selected from the group consisting of sepsis, a central nervous system injury, antibiotic resistance, compensatory anti-inflammatory response syndrome (CARS), chronic inflammatory response syndrome (CIRS), spinal cord injury-induced immune depression syndrome (SCI-IDS), traumatic brain injury-induced immune depression syndrome (TBI-IDS), combined CNS-injury induced immune deficiency syndrome (CIDS), and other immune deficiencies.
